(19) United States
(12) Patent Application Publication
(10) Pub. No.: US 2023/0149658 A1
(43) Pub. Date: May 18, 2023

(54) CONTROL METHOD FOR MEDICAL VENTILATORS

(71) Applicant: Imperial College Innovations Limited, London (GB)

(72) Inventors: Joseph van Batenburg-Sherwood, London (GB); Jakob Mathiszig-Lee, London (GB); Michael Madekurozwa, London (GB)

(21) Appl. No.: 17/917,413

(22) PCT Filed: Apr. 8, 2021
(86) PCT No.: PCT/EP2021/059237

§ 371 Date: Oct. 6, 2022

(30) Foreign Application Priority Data
Apr. 8, 2020 (GB) 2005249.4

(51) Int. Cl. A61M 16/20; A61M 16/00

(52) U.S. Cl. CPC A61M 16/205; A61M 16/024; A61M 2016/0042; A61M 2230/46

(57) ABSTRACT

A method of controlling exhalation in a ventilation system for providing Positive Expiratory End Pressure, PEEP, ventilation to a lung is disclosed, the method comprising: determining a lung resistance based on conditions of the system detected during an exhalation; and causing the system to inhibit system exhalation to cause and maintain a target system pressure based on the determined lung resistance and a pressure condition in the system.

### FIELD

The present disclosure relates generally to methods of controlling exhalation in medical ventilators. In particular, but without limitation, the present disclosure relates to methods of controlling and maintaining a Positive Expiratory End Pressure (PEEP) in lungs connected to medical ventilators.

### BACKGROUND

Patients in intensive care often require invasive ventilation such as positive pressure ventilation where air or another gas mix is delivered to the lungs. This process involves careful control of the pressure in the lung during both inhalation and exhalation. A positive pressure is required during exhalation in order to keep alveoli open, and thereby optimise oxygenation. During positive pressure ventilation, Positive Expiratory End Pressure (PEEP) is applied by the ventilator at the end of each breath to ensure the alveoli are not so prone to collapse. PEEP is the minimum pressure within the lungs occurring during exhalation. Varying levels of PEEP can be applied depending on the condition being treated.

The primary aims of ventilation (oxygenation and carbon dioxide clearance) require a balance between PEEP and minute volume. Minute volume is the total volume of gas breathed over a minute. A limiting factor for increasing minute volume is the time needed to exhale the tidal volume (the amount of gas in one breath). If a breath is not fully exhaled before the next breath starts (referred to as breath stacking), this can lead to hyperinflation and patient harm.

Conventional expiratory systems control PEEP by regulating air flow with a valve that provides varying resistance to flow. The simplest ventilators use a passive spring-loaded diaphragm system, with the resistance tuned by a clinician, to achieve the desired PEEP. For conventional systems with passive valves, a higher PEEP requires a higher resistance, and therefore increases the exhalation time. Increased exhalation time can lead to breath stacking if inhalation rate is maintained, or can lead to reduced ventilation if inhalation rate is decreased to allow for the increase in exhalation time.

More complex ventilators (of the type found in an Intensive Care Unit) use actively controlled proportional valves that can incrementally vary the extent of resistance in real-time, allowing a reduction in resistance to reduce exhalation time then an increase in resistance to provide the desired PEEP. Such ventilators improve performance significantly, but require complex and expensive expiratory valve subsystems. Ventilator systems comprising actively controlled proportional valves mitigate the problem of passive valves using complex control systems.

It is an object of the present invention to provide a method for controlling exhalation in ventilation systems for providing PEEP ventilation, which alleviates the problems of conventional systems. It is another object of the present invention to provide a method for controlling exhalation in ventilation systems for providing PEEP ventilation which enables reduced exhalation times, is employable with simple equipment in a cost effective way, and/or can be retro-fitted to existing ventilation systems.

### SUMMARY

Aspects and features of the present disclosure are defined in the accompanying independent claims.

A method of controlling exhalation in a ventilation system for providing Positive Expiratory End Pressure, PEEP, ventilation to a lung, the method comprising: determining a lung resistance based on conditions of the system detected during an exhalation; and causing the system to inhibit system exhalation to cause and maintain a target system pressure based on the determined lung resistance and a pressure condition in the system.

The conditions of the ventilation system may comprise data obtained in a first exhalation, and the determined lung resistance from the first exhalation may be used to cause the system to inhibit system exhalation in further exhalations.

The conditions of the system may comprise a system pressure condition and a system exhalation flowrate condition.

The system pressure condition may be based on a pressure differential between two system pressures, one measured before causing the system to inhibit system exhalation and one measured after causing the system to inhibit system exhalation.

The system pressure before causing the system to inhibit exhalation may be the system pressure measured at a system low pressure target, and the system pressure after causing the system to inhibit system exhalation may be the system pressure measured at a time when the system pressure equalises with a lung pressure as a consequence of causing the system to inhibit system exhalation.

The system low pressure target may be a target PEEP corresponding to the target system pressure.

The system flowrate condition may be based on a flowrate differential between two system flowrates, one measured before and one measured after causing the system to inhibit system exhalation. The flowrate measured after causing the system to inhibit system exhalation will be substantially zero.

The system flowrate before causing the system to inhibit system exhalation may be the exhalation flowrate measured at the system low pressure target (which may optionally be the target PEEP), and the system exhalation flowrate after causing the system to inhibit system exhalation may be the exhalation flowrate measured at a time when system pressure equalises with a lung pressure as a consequence of causing the system to inhibit system exhalation (which will be substantially zero).

The method may further comprise causing the opening of a valve thereby providing substantially no resistance to system exhalation prior to causing the system to inhibit system exhalation.

The system exhalation may be inhibited by causing the closing of a valve, optionally the closing of an on-off type valve or the closing of a proportional valve, configured to be in one of a fully closed position or a fixed open position.

The system exhalation may be inhibited by causing a single closing of the valve.

The valve provided may be configured to be in a fixed open position or a substantially fully closed position, said valve being in the open position during the exhalation apart from when system exhalation is inhibited and the valve is in the substantially fully closed position. That is, in carrying out the method the valve only moves between two fixed positions. System exhalation is inhibited when the valve is closed and in the fully closed position. Preferably the fixed open position is a position where the valve is substantially fully open (i.e. the valve is open to its fullest extent, providing substantially no resistance to system exhalation) however it may alternatively be e.g. 50-99% open.

Providing such a valve and operating it between only fixed open and fully closed positions advantageously provides an effective method for controlling exhalation in ventilation systems for providing PEEP ventilation providing reduced exhalation times (thereby reducing risk of breath stacking) and using less complex and cost effective equipment, e.g. on-off type valves, compared to prior art systems comprising actively controlled proportional valves. The methods disclosed herein may be used employing proportional valves operating in fixed open and substantially fully closed positions.

The method may further comprise providing a pressure sensor and using said sensor to determine the conditions of the system.

The method may further comprise repeating the determining and causing steps in subsequent exhalations.

The method may further comprise using in the repeated causing step(s) an averaged lung resistance as the lung resistance, said averaged lung resistance being based on an average of lung resistances determined from previous exhalations.

The method may further comprise a step of determining an error occurring during the exhalation in reaching the target system pressure caused by a timing delay in causing the system to inhibit system exhalation, and subsequently causing a timing correction (an offset) in causing the system to inhibit system exhalation to correct said error in a subsequent exhalation.

There is also provided an apparatus arranged to perform the methods described herein.

The apparatus may comprise a processor and a ventilation system configured to perform the methods described herein.

There is also provided a computer-readable medium carrying computer-readable instructions which, when executed by a processor of a ventilation system, cause the system to carry out the methods described herein.

### BRIEF DESCRIPTION OF THE DRAWINGS

Examples of the present disclosure will now be explained with reference to the accompanying drawings, in which:

FIG. 1 shows an arrangement in which a controller is operable to control a controlled system;

FIG. 2 shows a block diagram of a controller;

FIG. 3 shows a flow chart of the steps of a method of control;

FIG. 4 shows ventilation exhalation pressure and flow curves for a ventilation system where exhalation is controlled by a method of this disclosure; and

FIG. 5 shows ventilation exhalation pressure and flow curves for a ventilation system controlled by a method of this disclosure.

### DETAILED DESCRIPTION

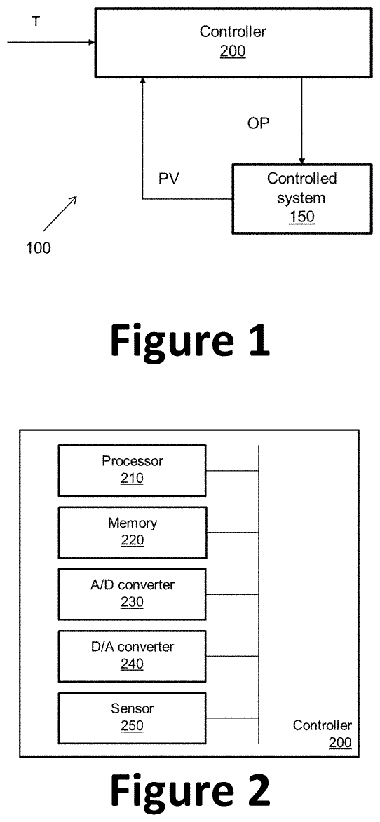

FIG. 1 shows a control arrangement 100 comprising a controlled system 150 (or ‘process’), which is controlled by a controller 200. The controller 200 receives targets T as inputs, and outputs a control signal OP, which is fed to the controlled system 150. Conditions (or ‘variables’) of the controlled system 150 are determined, and fed back to the controller 200 to be used in the determination of the control signal OP. In this way, a feedback loop may be established, thereby enabling accurate control of the controlled system 150.

The controlled system 150 may be a medical ventilation system, such as a ventilation system for providing PEEP control comprising at least a valve (or ‘exhalation valve’) to control exhalation resistance in the system, a ventilation chamber for providing PEEP control, and system pressure and flowrate sensors. In that case, the input targets T may be indicative of a target pressure (such as a ‘target PEEP’), a low pressure target (such as ‘a low pressure PEEP target’ or ‘−ΔPEEP’) and a high pressure target (such as ‘a high pressure PEEP target’ or ‘+ΔPEEP’). These input targets may act as triggers to cause system changes discussed herein and therefore may be referred to as ‘triggers’. The conditions may be indicative of a measured system pressure and system outlet flowrate. System outlet flowrate is controlled by the exhalation valve (i.e., the flowrate is determined by the state of the exhalation valve), and relates to the outlet fluid flowrate (which comprises a mix of predominantly air and oxygen (added oxygen being present from the ventilation process)) from the ventilation system as a result of a lung exhalation. System outlet flowrate may be determined by the pressure drop occurring across the exhalation valve (and with knowledge of the flow-pressure characteristics of the exhalation valve in its fixed open state). The exhalation valve is capable of inhibiting (i.e. ‘fully preventing’, or ‘stopping’) the flowrate of air out of the ventilation system, and can be (and is preferably) an on-off valve.

If an on-off valve is part of the system then the valve is either fully open (i.e. does not inhibit air flowrate out of the ventilation system) or is fully closed (i.e. substantially fully inhibits flowrate out of the ventilation system) and the method provides effective and active PEEP control based on a single fixed resistance (provided by the binary nature of the on-off valve). A proportional valve may be used instead of an on-off type valve to a similar effect, by configuring it to operate in a fixed open position (which is preferably substantially fully open but may be e.g. 50-99% fully open) or a substantially fully closed position.

FIG. 2 shows a block diagram of the controller 200, which may be used for implementing elements of the methods described herein. The controller 200 comprises a processor 210 arranged to execute computer-readable instructions, which may be stored in a memory 220, for example a random access memory. The memory 220 may also store previous values of any of the signals described below. The processor 210 may receive data, e.g., conditions from the controlled system 150 and the targets T, via an analog-to-digital (A/D) converter. The processor 210 may also output data, e.g., the control signal OP, via a digital-to-analog (D/A) converter. A sensor 250 may be arranged to determine the state variable PV of the controlled system 150 and to communicate that state variable to the A/D converter 230 and/or to the processor 210. Although the controller 200 of FIG. 2 comprises a computer processor, a person skilled in the art will understand that the methods described herein may alternatively be implemented using analog circuitry.

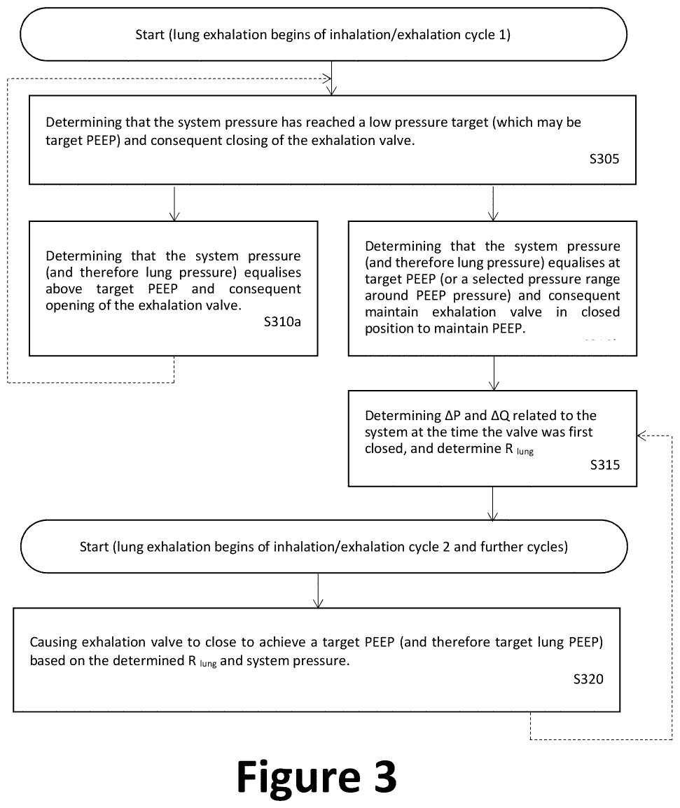

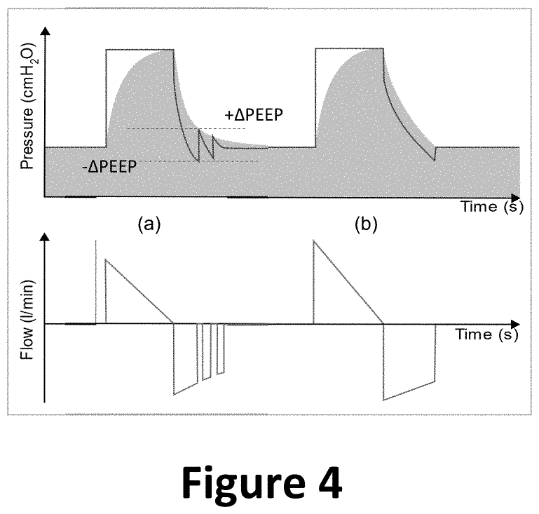

A method of control of the controlled system 150 is explained with respect to FIGS. 3 and 4. This method may be implemented by the controller 200, or indeed by any processor or processing means.

FIG. 4 shows two lung inhalation/exhalation cycles (cycle (a) and cycle (b)), the exhalation aspects of which being controlled using the method disclosed herein. The exhalation aspect of one inhalation/exhalation cycle may be referred to as ‘an exhalation’. The lines on the pressure charts show the pressure in the ventilation system (the pressure in the pipework/tubing on the exhalation side of the ventilation system), and the shaded area indicates approximate pressure in the non-conducting airways of the lung (or ‘lung pressure’). Each cycle (a) and (b) is visible as a rise in pressure due to a lung inhalation, followed by a decrease in pressure due to lung exhalation.

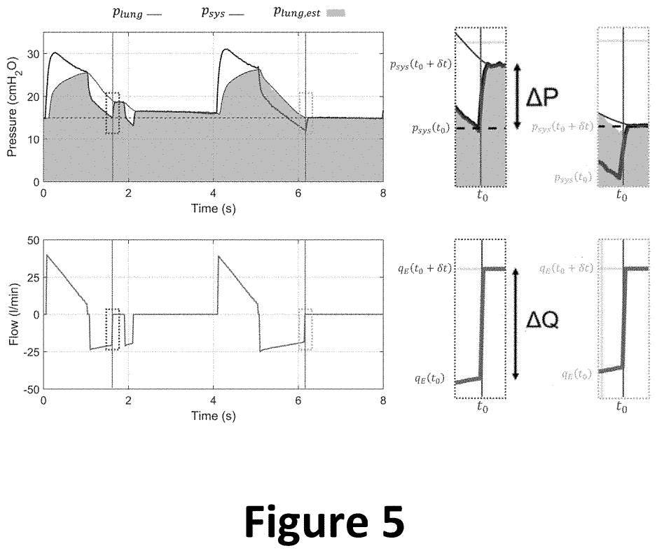

Step S305 occurs during an exhalation. During an exhalation, system pressure decreases rapidly as the exhalation valve is open providing substantially no exhalation resistance. Step S305 involves determining that the system pressure has reached the low pressure PEEP target (shown as −ΔPEEP in FIG. 4 in relation to cycle (a), but may alternatively be target PEEP as shown in FIG. 5) and consequently closing the exhalation valve. Before the valve closes, the grey shaded area on FIG. 4 shows that the pressure in the lung is higher than in the system—this is due to the pressure drop resulting from the lung resistance as well as resistances from the endotracheal tubes and the filter at the patient connector.

Closing the exhalation valve in step S305 results in fluid flow out of the ventilation system to be stopped, and the system pressure rapidly increases to match the lung pressure as the lungs continue to exhale thereby pressurising (and equalising with) the system. Step S310a therefore involves determining when the system pressure increases and meets a predefined high pressure PEEP target (shown as +ΔPEEP in FIG. 4 in relation to cycle (a)), and consequently opening the exhalation valve thereby depressurising the system. Before the exhalation valve closes the pressure in the lung is higher than in the system due to the pressure drop across the lung (as a result of lung resistance as well as resistances from the endotracheal tube and the filter at the patient connector). These processes are repeated until a stable system pressure matching target PEEP (or a selected pressure range substantially near PEEP pressure, e.g. +/−10% target PEEP) is provided and maintained (this repetition in steps S305 and S310a is represented by a dotted line in FIG. 3). The target pressure is maintained by keeping the exhalation valve closed (i.e. by inhibiting system exhalation) for the time desired before the inhalation of the next inhalation/exhalation cycle. In other words, steps S310a and S310b involve stabilising (or ‘equalising’) system pressure with lung pressure at the target PEEP. This process of stabilising results in oscillating system pressures as the system pressure stabilises, which increases exhalation time. Step S315 addresses the problem of oscillating system pressure while the system pressure stabilises.

In step S315, the change in pressure and flowrate that occurs when the exhalation valve first closes at the low pressure target (discussed above in relation to the rapid pressure increase in step S305) is used to determine lung resistance (R_{lung}). R_{lung} is a function of change in pressure (ΔP) and flowrate (ΔQ) R and for an organic lung (e.g. a human or animal lung) may be expressed as R_{lung}=ΔP/Q (For step S315 ΔQ=Q since flowrate is reduced to zero when the exhalation valve is closed). Lung resistance defined herein also includes resistances provided by the endotracheal tube, filters at the patient connectors and any other breathing system apparatus, but in the context of providing PEEP ventilation lung resistance typically dominates these resistances hence defining the resistance as lung resistance herein, although it could alternatively be referred to as ‘airway resistance’ and have the same meaning.

Accordingly, when the exhalation valve first closes the ΔP and Q are captured. With these conditions obtained, R_{lung} may then be determined and applied to the next inhalation/exhalation cycle in step S320; during step S320 of the second cycle (and further cycles), true lung pressure can be determined based on system pressure and R_{lung}; and the controller may then only cause (instruct) the exhalation valve to close when, upon closing, the system pressure and lung pressure will equalise on the target PEEP (i.e. the lung reaches the target PEEP) as shown in the cycle (b) of FIG. 4. With R_{lung} known, the pressure in the lung can be predicted in real-time thereby allowing the exhalation valve (e.g. an on-off valve) to shut only once, at the point where, upon equalising with the system pressure, the pressure in the lungs will reach the target PEEP. Closing the exhalation valve once only when necessary in this way reduces exhalation time in high resistance airways, drastically reduces wear that would otherwise be incurred by the valve (which typically have a finite number of changing cycles before they stop working), and reduces the number of starts/stops in exhalation flow experienced by a patient's lungs.

Knowing the lung pressure that will result after closing the valve during the second and further exhalations assists in determining when to cause the valve in step S320 to close. A way of determining the lung pressure that will result following closure of the exhalation valve will now be described.

During exhalation, lung pressure (P_{lung}) can be determined by the following equation 1:

P_{lung}=P_{sys}+R_{lung}Q (equation 1)

where P_{sys} is system pressure and Q is flow rate of the exhalation flow from the system.

The exhalation starts with the exhalation valve opening. To achieve accurate PEEP control, the ideal behaviour is for the exhalation valve to close when P_{lung} reaches target PEEP. P_{lung} cannot be directly measured and instead must be estimated according to the following equation 2 to give P_{lung, est} as a function of R_{lung}, P_{sys}, and Q.

P_{lung,est}=P_{sys}+R_{lung}Q (equation 2)

‘P_{sys}’ is directly monitored as the pressure at the exhalation valve and ‘Q’ may be determined from data obtained from the P_{sys} data, and if so is calculated based on the following equation 3:

Q=a(P_{sys}−Pₐₜₘ)ⁿ (equation 3)

where ‘a’ and ‘n’ are constants calculated by calibration.

The value of R_{lung} is dependent on the specifics of the patient's lungs and other factors such as the size of the endotracheal tube, the amount of secretions in the system etc., so it cannot be calculated a priori and will change with time.

Initially, during the first exhalation (cycle 1) it is assumed that R_{lung} is equal to zero, hence based on equation 2 the estimated lung pressure (P_{lung})=P_{sys} during exhalation (note again that this is for the first breath, and all subsequent exhalation cycles use determined R_{lung, est} value to determine R_{lung}).

During step S305 the exhalation valve closes at time to when P_{lung}=−ΔPEEP (which may alternatively be target PEEP (P_{PEEP})), and hence:

P_{lung}(t₀)=P_{sys}(t₀)R_{lung}Q(t₀) (equation 4)

A very short time (δt) after the valve closes (time t+δt), we have:

P_{lung}(t₀+δt)=P_{sys}(t₀+δt)+R_{lung}Q(t₀+δt) (equation 5)

Once the exhalation valve is closed the flow rate becomes zero, i.e. Q(t₀+dt)=0, and the assumption is taken that:

P_{lung}(t₀+δt)=P_{lung}(t₀) (equation 6)

This assumption is taken because the pressure in the lung is determined by its compliance and the instantaneous volume in the lung. As there is negligible change in the lung volume in the time it takes to close the exhalation valve, there is correspondingly a negligible change in lung pressure.

Combining equations 4, 5, and 6 yields equation 7 which is used in step S315:

$${R}_{lung} = \frac{{P}_{sys} ( {t}_{0} + δ t ) - {P}_{sys} ( {t}_{0} )}{Q ( {t}_{0} )} \qquad ( equation 7 )$$

The value of R_{lung} can then be used in equation 2 for step S320 during a subsequent breath to calculate P_{lung, est} which can be used to cause the exhalation valve to close when P_{lung, est}=target PEEP, i.e. causing exhalation valve to close to achieve a target PEEP (and therefore target lung PEEP) based on the determined R_{lung} and system pressure (P_{sys}).

The value of R_{lung} calculated with equation 7 is preferably R_{lung}(Q(t₀)). Correspondingly, the estimated lung pressure (P_{lung,est}) preferably corresponds to the actual lung pressure when Q=Q(t₀). As R_{lung} could be expected to scale approximately linearly with Q, using values of R_{lung} calculated at arbitrary flow rates would result in less accurate estimates of P_{lung,est} and hence the exhalation valve would close too early or too late. R_{lung} could be calculated at any time in the exhalation breathing cycle, however, the value of R_{lung} calculated would be different to the value of R_{lung} needed to close the exhalation valve at target PEEP.

The steps S305 to S315 of this method are primarily described so far as steps which performed at the beginning of a ventilation process, i.e. during the exhalation of a first inhalation/exhalation cycle in a series of inhalation/exhalation cycles, to calibrate the system to the specific ventilation system being used (based on the tubing and other aspects of the ventilation system) and the resistance of the lung being ventilated. However, the steps of this method may be steps that are performed repeatedly i.e., in an iterative manner, or at predefined time intervals. As a switch still occurs when the valve closes in step S320, the lung resistance can be continually monitored to account for dynamics changes and step S320 may be carried out based on each given preceding breath, or based on averaged R_{lung} values averaged over two or more exhalation breath cycles. This continuous monitoring and implementing of changes dynamically is shown by the dashed line from step S320 back to step S315.

In cases of high lung resistance and/or low lung compliance and/or where there are lag times between system signalling, the response time of the controlled system can result in a system reaction that is too slow, resulting in a PEEP that is too low (i.e. below the target PEEP). To correct this, the control system can measure the degree in which PEEP is too low (εPEEP), and on the subsequent inhalation/exhalation cycle the exhalation valve can be triggered to close when the estimated lung pressure reaches PEEP+εPEEP, which successfully accounts for the slow reaction on the subsequent breath by a ‘predefined time interval’. A similar response time correction may be applied if the system reaction is too fast, i.e. if the valve is caused to close resulting in a PEEP above the target PEEP.

The methods described herein allow the passive spring-loaded diaphragms of known ventilation systems to be replaced with a simple on-off type valve as the exhalation valve to control exhalation whilst reducing exhalation time, maintaining the desired PEEP, and avoiding the requirement of use expertise to operate the system accurately. Exhalation time is reduced since, until system exhalation is caused to stop, substantially no exhalation resistance is provided when the exhalation valve is fully open (the minimal resistance that exists being provided by internal components of the ventilator system such as tubing/pipework and open valves). Having substantially no resistance (i.e. a small amount of resistance provided by internal components of the ventilator system) has been found to be beneficial—if there was no resistance, exhalation would be instantaneous, and it would be very difficult to control PEEP or measure flow as described herein. Having a small degree of resistance to exhalation provides improved exhalation times without being detrimental to system control performance. The methods disclosed herein cause the valve (e.g. an on-off valve) to close once, in order to cause the system and lung pressure to simultaneously reach the target PEEP. The methods described herein avoid the requirement for use of complex equipment and control systems associated with known methods for controlling ventilator expiration actively involving incrementally varying the extent of exhalation resistance in real-time with complex and expensive expiratory valve subsystems.

FIG. 5 shows experimental data obtained using a PEEP ventilator system and where a method of this disclosure was used to control the exhalation part of a PEEP ventilator. FIG. 5 shows how ΔP and ΔQ measurements are taken from a first inhalation/exhalation cycle for use in determining R_{lung}. FIG. 5 shows the application of step S320 for the second inhalation/exhalation cycle (applying the determined R_{lung} and known system pressure to close the exhalation valve at the point where the system and lung are in equilibrium at the target PEEP on the exhalation of the second cycle).

The lung test shown in FIG. 5 is an artificial lung comprising a series resistance and compliance, where R_{lung}=Q/Kᵥ². Valve resistance increases with flowrate due to turbulence and can be represented by the relationship Kᵥ=ΔP^{0.5}/ΔQ where Kᵥ is a flow factor. For a real lung, resistance may be insensitive to flowrate, e.g. R_{lung}=ΔP/ΔQ. The lung tested in FIG. 5 comprises a Kᵥ initially set to 100 (m³/hr/bar^{0.5}), hence assuming negligible resistance on the exhalation, and the reason why the predicted lung pressure (P_{lung,est}) (shaded grey) and system pressure (line, P_{sys}) are equal on the first exhalation cycle. Actual lung pressure (R_{lung}) is shown in FIG. 5 as the red line which does not equal P_{sys} on the exhalation cycles.

The methods described herein may be embodied on a computer-readable medium, which may be a non-transitory computer-readable medium. The computer-readable medium carries computer-readable instructions arranged for execution upon a processor so as to make the processor carry out any or all of the methods described herein.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

The above description has been made in terms of specific examples for the purpose of illustration and not limitation. Many modifications and combinations of, and alternatives to, the features described above will be apparent to a person skilled in the art and are intended to fall within the scope of the invention, which is defined by the claims that follow.

1. A method of controlling exhalation in a ventilation system for providing Positive Expiratory End Pressure, PEEP, ventilation to a lung, the method comprising:
determining a lung resistance based on conditions of the system detected during an exhalation; and
causing the system to inhibit system exhalation to cause and maintain a target system pressure based on the determined lung resistance and a pressure condition in the system.

2. The method of claim 1, wherein the conditions of the ventilation system comprise data obtained in a first exhalation, and wherein the determined lung resistance from the first exhalation is used to cause the system to inhibit system exhalation in further exhalations.

3. The method of claim 1, wherein the conditions of the system comprise a system pressure condition and a system exhalation flowrate condition.

4. The method of claim 3, wherein the system pressure condition is based on a pressure differential between two system pressures, one measured before causing the system to inhibit system exhalation, and one measured after causing the system to inhibit system exhalation.

5. The method of claim 4, wherein the system pressure before causing the system to inhibit exhalation is the system pressure measured at a system low pressure target, and the system pressure after causing the system to inhibit system exhalation is the system pressure measured at a time when the system pressure equalises with a lung pressure as a consequence of causing the system to inhibit system exhalation.

6. The method of claim 5, wherein the system low pressure target is a target PEEP which corresponds to the target system pressure.

7. The method of claim 5, wherein the system flowrate condition is based on a flowrate differential between two system flowrates, one measured before and one measured after causing the system to inhibit system exhalation.

8. The method of claim 7, wherein the system flowrate before causing the system to inhibit system exhalation is the exhalation flowrate measured at the system low pressure target, and the system exhalation flowrate after causing the system to inhibit system exhalation is the exhalation flowrate measured at a time when system pressure equalises with a lung pressure as a consequence of causing the system to inhibit system exhalation.

9. The method of claim 1, further comprising causing the opening of a valve thereby providing substantially no resistance to system exhalation prior to causing the system to inhibit system exhalation.

10. The method of claim 1, wherein the system exhalation is inhibited by causing the closing of a valve, optionally the closing of an on-off type valve or the closing of a proportional valve configured to be in one of a fully closed position or a fixed open position.

11. The method of claim 10, wherein the system exhalation is inhibited by causing a single closing of the valve.

12. The method of claim 9, wherein the valve provided is configured to be in a fixed open position or a substantially fully closed position, said valve being in the open position during the exhalation apart from when system exhalation is inhibited and the valve is in the substantially fully closed position.

13. The method of claim 12, wherein the fixed open position is substantially fully open.

14. The method according to claim 1, further comprising providing a pressure sensor and using said sensor to determine the conditions of the system.

15. The method of claim 1, further comprising repeating the determining and causing steps in subsequent exhalations.

16. The method of claim 15, further comprising using in the repeated causing step(s) an averaged lung resistance as the lung resistance, said averaged lung resistance being based on an average of lung resistances determined from previous exhalations.

17. The method of claim 1, further comprising determining an error occurring during the exhalation in reaching the target system pressure caused by a timing delay in causing the system to inhibit system exhalation, and subsequently causing a timing correction in causing the system to inhibit system exhalation to correct said error in a subsequent exhalation.

18. An apparatus arranged to perform the method of claim 1.

19. An apparatus of claim 18, wherein the apparatus comprises:
a processor and a ventilation system configured to perform the method of claim 1.

20. A computer-readable medium carrying computer-readable instructions which, when executed by a processor of a ventilation system, cause the system to carry out the method of claim 1.
